(19) United States
(12) Patent Application Publication
(10) Pub. No.: US 2019/0254805 A1
(43) Pub. Date: Aug. 22, 2019

(54) BIFURCATED STENT GRAFT WITH HEMODYNAMIC BLOOD FLOW DIVIDING WALL

(71) Applicant: Cook Medical Technologies LLC, Bloomington, IN (US)

(72) Inventors: Ralf Spindler, Bloomington, IN (US); Georgios Hilas, Bloomington, IN (US)

(21) Appl. No.: 16/399,017

(22) Filed: Apr. 30, 2019

Related U.S. Application Data
(63) Continuation-in-part of application No. 15/818,023, filed on Nov. 20, 2017.
(60) Provisional application No. 62/452,622, filed on Jan. 31, 2017.

(51) Int. Cl. A61F 2/07

(52) U.S. Cl. CPC A61F 2/07; A61F 2002/065

(57) ABSTRACT

A bifurcated stent graft includes a stent graft body that defines exactly one main body opening and two exit openings. The stent graft body includes at least one stent attached to a graft fabric material, and includes a dividing wall that divides a combined flow path, into a first flow path and a second flow path that each terminate at one of the respective exit openings. The dividing wall includes a thickness profile that is equal to or thinner than a wall thickness of the graft fabric material, terminates a leading rounded surface, and extends across a width of the combined flow path.

### TECHNICAL FIELD

The present disclosure relates generally to bifurcated stent grafts, and more particularly to a dividing wall that terminates at a leading rounded surface to improve blood flow dynamics.

### BACKGROUND

In some instances, abdominal aortic aneurism repair is accomplished by implanting a bifurcated stent graft that includes a combined flow path portion that spans the aneurism, and a pair of leg portions that are respectively received in the left and right iliac arteries. Although these devices have performed well for many years, researchers have observed at least one major drawback can result in an increased risk of long term complications for a patient. In particular, it is believed that blood flow dynamics, especially in the vicinity of the bifurcation of the stent graft, can cause blood cell damage and blood protein conformational changes that can lead to complications. In most applications, the bifurcation consists of a saddle area where the stent graft portions for the two iliac arteries are joined.

The present disclosure is directed toward one or more of the problems set forth above.

### SUMMARY

In one aspect, the bifurcated stent graft includes a stent graft body that defines exactly one main body opening and two exit openings. The stent graft body includes at least one stent attached to a graft fabric material, a bifurcation, and a dividing wall that divides a combined flow path into a first flow path and a second flow path that each terminate at one of the respective exit openings. The dividing wall includes a thickness profile that is equal to or thinner than a wall thickness of the graft fabric material, terminates a leading rounded surface, and extends across a width of the combined flow path.

### BRIEF DESCRIPTION OF THE DRAWINGS

FIG. 1 is a perspective view of a bifurcated stent graft according to the present disclosure;

FIG. 2 is a sectioned view through the bifurcated stent graft of FIG. 1;

FIG. 3 is a sectioned view through the bifurcated stent graft of FIG. 1 as viewed along section lines 3-3 of FIG. 2;

FIG. 4 is a sectioned view through a portion of a dividing wall for the bifurcated stent graft of FIGS. 1-3;

FIG. 5 is a sectioned view through a dividing wall for a bifurcated stent graft according to another embodiment of the present disclosure;

FIG. 6 is a schematic view for illustrating geometrical relationships between the dividing wall and the underlying stent graft body geometry;

FIG. 7 is a schematic view of a bifurcated stent graft illustrating an extreme short height dividing wall aspect of the present disclosure;

FIG. 8 is a schematic view of still another bifurcated stent graft showing an extreme tall version of the dividing wall aspect of the present disclosure;

FIG. 9 is a schematic view of a bifurcated stent graft according to another aspect of the present disclosure;

FIG. 10 is a sectioned view of the bifurcated stent graft of FIG. 9 as viewed along sectioned lines 10-10;

FIG. 11 is a sectioned view of the bifurcated stent graft of FIG. 9 as viewed along sectioned lines 11-11;

FIG. 12 is a sectioned view through the bifurcated stent graft of FIG. 9 as viewed along sectioned lines 12-12;

FIG. 13 is a sectioned view of the bifurcated stent graft of FIG. 9 as viewed along sectioned lines 13-13;

FIG. 14 is an enlarged partial sectioned view of the dividing wall structure according to the embodiment of FIG. 9;

FIG. 15 is a schematic sectioned view through the graft fabric material for thickness comparison with the dividing wall shown in FIG. 14;

FIG. 16 is a schematic view of a dividing wall showing a continuum of decreasing permeability through the wall;

FIG. 17 is a schematic view of a dividing wall showing step wise decreases in permeability through the wall; and

FIG. 18 is a sectioned view through a dividing wall showing a continuum of permeability change according to another aspect of the present disclosure.

### DETAILED DESCRIPTION

Referring to all of the Figs., a bifurcated stent graft 10, such as a stent graft utilized in abdominal aortic aneurism repair, includes a stent graft body 11 that defines exactly one main body opening 12 and two exit openings 13, 14. The stent graft body 11 includes at least one stent 15 attached to a graft fabric material 16. In one specific example, the stent 15 is a self expanding stent of a type well known in the art, and the graft fabric material 16 can be any suitable material known in the art. From the outside, bifurcated stent graft 10 may look much like commercially available bifurcated stent grafts that have been known and used with considerable success for years. However, a stent graft body 11 according to the present disclosure includes a dividing wall 20 (120, 220, 320, 420, 520) that divides a combined flow path 17 into a first flow path 18 and a second flow path 19 that each terminate at one of the respective exit openings 13, 14. The dividing wall includes a thickness profile 21 that terminates at a leading edge radius or rounded surface 22 (122, 222, 322, 422, 522), and the dividing wall extends across a width 25 of the combined flow path 17. Thus, the present disclosure teaches dividing the flow destined for the iliac arteries starting at the leading edge radius or rounded surface 22 (122, 222, 322, 422, 522) of an internal dividing wall 20 (120, 220, 320, 420, 520), rather than at the crotch or bifurcation 37 of the graft 10 as per the prior art.

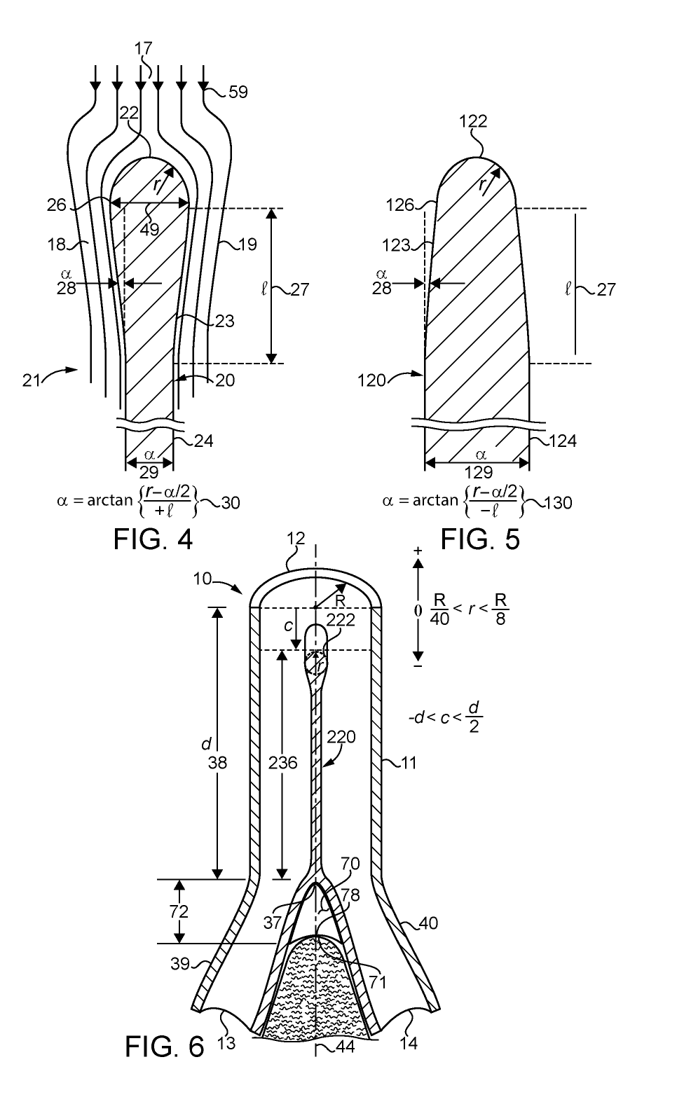

The thickness profile 21 of the dividing wall 20 can have a variety of confirmations and still fall within the scope of the present disclosure. For instance, thickness profile 21 may include a tapered segment 23 that smoothly transitions into the leading edge radius 22 at a tangent. The tapered segment 23 may begin as a tangent 26 to the leading edge radius or rounded surface 22. Apart from the possible inclusion of a taper segment 23, the thickness profile 21 of the dividing wall 20 may also include a uniform thickness segment 24. In this context, “uniform thickness” means that this segment has no evident taper, but may show some variability due to the underlying materials (e.g. metallic stenting). The dividing wall 20 may be formed at least partially of the same material as the graft fabric material 16 and may stretch between opposite sides of the stent graft body 11, and may or may not include metallic stent or other rigid skeleton (e.g., PTFE) support and/or reinforcement. The leading edge radius 22 as well as the contiguous tapered segment 23 or uniform thickness segment 24 may be also formed from the graft fabric material 16, and or may also include a formed feature, such as from plastic, to shape the thickness profile 21 in general, and the leading edge radius or rounded surface 22, in particular. Other materials could also be considered (and are described infra) so that dividing wall 20 is thinner than a thickness of the graft fabric material 16 so as to minimize profile addition in the crimped state (not shown). The tapered segment 23 may have a length 27 (1) greater than double a radius r of the leading edge radius 22. In two specific examples, shown in FIGS. 4 and 5, respectively, a taper angle 28 (α) of the tapered segment 23, 123 may be a function 30, 130 of the radius r of the leading edge radius 22, 122, the length 27 (l) of the tapered segment 23, 123 and a thickness 29, 129 (a) of the uniform thickness segment 24, 124. As shown in FIG. 4, the tapered segment 23 may thin toward the leading edge radius 22, or as shown in FIG. 5, the tapered segment 123 may thicken toward leading edge radius 122. The location that blood first encounters the leading edge radius 22, 122 can be considered a leading rounded surface according to the present disclosure. Thus, the leading rounded surface may extend the entire width of leading edge radius 22, but the leading rounded surface occurs adjacent the stent graft side walls for leading edge radius 122 (see FIG. 3). This is because a leading edge radius 22 is perpendicular to blood flow, but leading edge radius 122 has a concave shape across width 25.

The dividing wall 20 has a height 36 that extends from the bifurcation 37 of the stent graft body 11 to the leading edge radius 22. In the solid line illustrated embodiment of FIG. 2, the height 36 is a minority of a distance 38 from the main body opening 12 to the bifurcation 37. Nevertheless, the present disclosure also contemplates a dividing wall height 136 corresponding to a leading edge radius 122 that is about half the distance 38 from the main body opening 12 to the bifurcation 37. About half means that when the ratio of the height 136 to the distance 38 is rounded to a fraction with a 1 in the numerator, then the denominator, when rounded to a single significant digit, is 2. In still another embodiment, the designer may opt to have the flow divide even closer to the main body opening 12. For instance, a dividing wall height 236 may be a majority of the distance 38 from the main body opening 12 to the bifurcation 37. A leading edge radius 222 positioned at the main body opening 12 would also be considered as dividing the combined flow path 17, and would still fall within the scope of this disclosure. The height 236 of the dividing wall 20 may be longer than the first leg 39 and the second leg 40 or, the height 36 of the dividing wall 20 may be shorter than both the first leg 39 and the second leg 40. Or, the height 136 of the dividing wall 120 may be about equal to a length of one of the legs 39 or 40. About equal means that when the two lengths are ratioed, and rounded to a single significant digit, that number is one.

The present disclosure also contemplates different leading edge shapes across a width 25 of the combined flow path 17. For instance, the solid line illustrated embodiment (FIG. 2) shows a straight line 32 across the width 25 that is oriented perpendicular to a blood flow direction. The dashed line for the leading edge radius 122 shows that the leading edge radius 122 may be presented as a curved line 31 across the width 25, which is concave to the flow path. In still another example, the leading edge radius 222 may have another concave shape with a center 233 of the width 25 that is further from the main body opening 12 than the two sides 234 and 235 of the width 25. Any of these different width shapes of the leading edge radius (22, 122, 222) can go with any of the different wall heights 36, 136, 236 without departing from the present disclosure. Other leading edge width profiles, including a convex shape, such as leading edge rounded surface or radius 422, would also fall within the intended scope of the present disclosure.

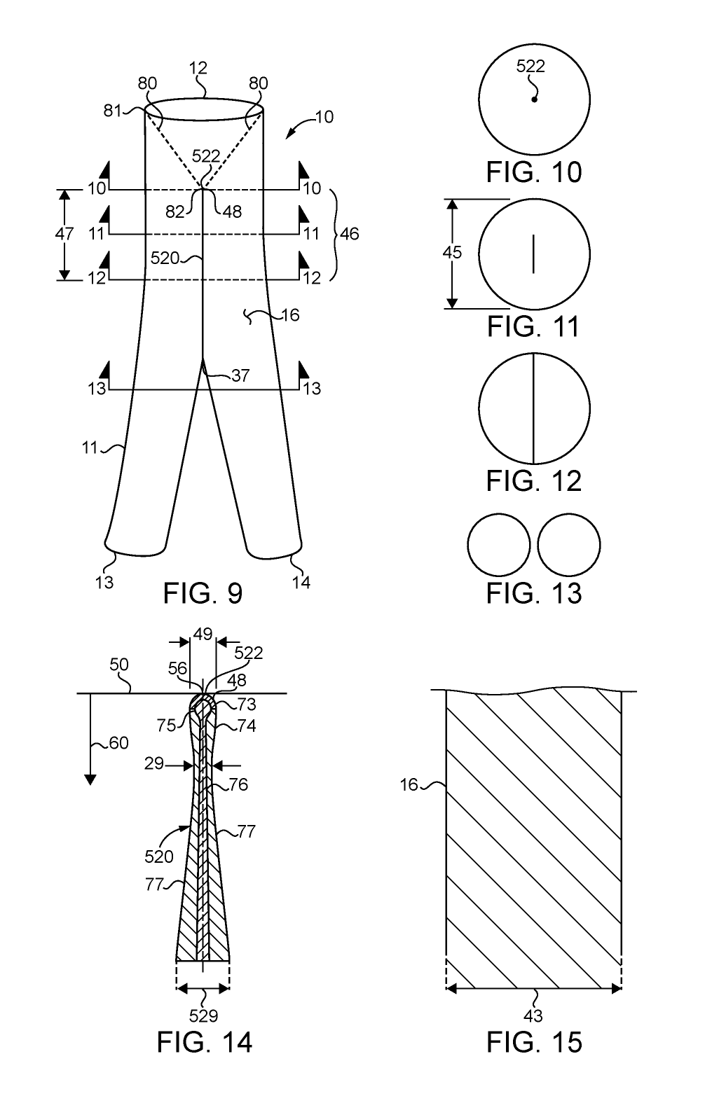

Although FIGS. 4 and 5 suggest that the cross sectional view of the tapered segment 23 and the leading edge radius 22 may be uniform across width 25, this need not necessarily be so, and still fall within the intended scope of the present disclosure. See e.g., FIGS. 9-13 discussed infra. Although the radius r may be different at different locations across the width 25, the blood flow will still be split at a leading edge radius 22, 122, 222, 322, 422, 522 that more atraumatically and efficiently splits the flow prior to arrival at the iliac arteries. Although the present disclosure is illustrated in the context of a bifurcated stent graft 10 in which one of the two exit openings 13 is further from the main body opening 12 than the other of the two exit openings 14, any bifurcated stent graft could fall within the scope of the present disclosure. Thus, in the illustrated embodiment the stent graft body 11 has a first leg 39 that extends from the bifurcation 37 to the exit opening 13, and a second leg 40 that extends from the bifurcation 37 to the other exit opening 14. A bifurcated stent graft according to the present disclosure need not necessarily be of the type typically associated with abdominal aortic aneurism repair.

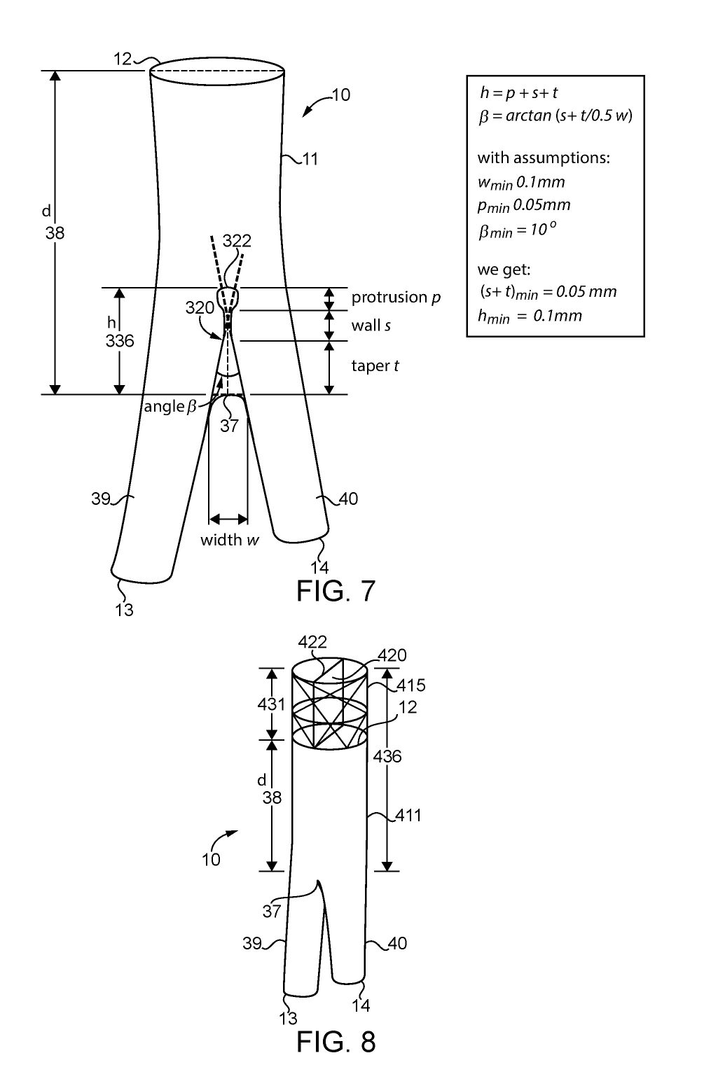

Referring now specifically to FIG. 6, which is not to scale, a schematic view of a bifurcated stent graft 10 is used to illustrate a range of relationships between the radius r of the dividing wall 220 to the geometry of the stent graft body 11. In particular, where R is the radius of the main body opening 12 and r is the radius of the leading edge radius 222, an equation expressing the range of the leading edge radii to the radius R of the main body opening is shown in FIG. 6. Also, FIG. 6 is useful in illustrating a relationship between the distance c between the main body opening 12 and the tip of the leading edge radius 222 in relation to the distance d from the main body opening 12 to the bifurcation 37. Thus, in one extreme (FIG. 7), the dividing wall 320 can be very short but does have some height from bifurcation 37. On the other hand, in another extreme case as shown in FIG. 8, the dividing wall 420 may extend outside of the main stent graft body 11 and beyond or upstream from the main graft opening 12. In such an instance, the extended portion of the dividing wall 420 that extends above main body opening 12 may be supported by bare metal stent structure 415 that may be uncovered or covered by any fabric 16 as in the stent graft body 11. The height 431 of the dividing wall 420 that extends above main body opening 12 may be about half the distance d 38 from the main body opening 12 to the bifurcation 37 as per the equation expressed in FIG. 6. Thus, in one extreme case illustrated in FIG. 8, the overall height 436 of the dividing wall 420 may be longer than the distance d 38 from the main body opening 12 to the bifurcation 37 and still fall within the scope of the present disclosure.

FIG. 6 also of interest for showing that stent grafts 10 according to the present disclosure may also include a stretchable membrane 70 that extends between legs 39 and 40 to include a contact surface 71 that is positioned a separation distance 72 from bifurcation 37. Stretchable membrane 70 may be attached to legs 39 and 40 in any suitable manner, such as by sutures. Contact surface 71 of stretchable membrane 70 may contact the aortic bifurcation 78 of the patient. This structure may inhibit rubbing interaction between the stent graft body 11 interaction with any calcification that may be formed at or near aortic bifurcation 78. Stretchable membrane 70 should be sufficiently pliable to permit legs 39 and 40 to be spread to accommodate any anatomy, yet be stiff enough to inhibit contact between the patients aortic bifurcation 78 and the bifurcation 37 of the stent graft 10. Preferably, stretchable membrane 70 is oriented perpendicular to the dividing wall 220.

Referring to FIG. 7, a set of equations and dimensions are provided for illustrating an extreme short height version of a dividing wall 320 according to the present disclosure. In particular, the illustration of FIG. 7 is extremely out of scale to show the geometry, but the numbers in the box to the right hand side show that the dimensions can be relatively small. In particular, the height h 336 of the dividing wall 320 is the sum of the height of the protrusion portion p that includes the leading edge radius 322, the height of wall s, which was earlier referred to as a uniform wall segment, and the height of taper t. The dividing angle β in one extreme is calculated as the arc tangent of s+t over ½ a width w of the bifurcation 37. Assuming that the bifurcation width is 0.1 millimeter, that the height of protrusion p is 0.5 millimeters and that the dividing angle β is 10°, we arrive at a minimum height for the dividing wall 320 as being 0.1 millimeters. Thus, the present disclosure contemplates dividing walls that extend from a fraction of a millimeter above the bifurcation 37 all the way to dividing walls that are actually longer than the length d of the main body segment of the bifurcated stent graft 10.

A variety of different structures are considered for the dividing wall 20 (120, 220, 320, 420, 520). Among these, the leading edge radius 22 (122, 222, 322, 422, 522) could be a coated or an un-coated polymer, such as PTFE, and/or the leading edge radius 22 (122, 222, 322, 422, 522) could be coated with an elastic (soft) layer to reduce cellular stress that might occur when the blood cells impact the leading edge radius 22 (122, 222, 322, 422, 522). This soft coating, which could be polyvinyl alcohol, may have a Young's Modulus from 1-5 MPa, or be biomimetically similar to endothial tissue. A lower Young's Modulus than natural tissue (e.g., 0.1 MPa) may also be desirable. Apart from being soft, the leading edge radius 22 (122, 222, 322, 422, 522) should be smooth to increase the likelihood of laminar blood flow. The leading edge radius 22 (122, 222, 322, 422, 522) may be portions of a cap made from a biomimetic material that mimics a hardness of an aortic bifurcation.

The dividing wall 20 (120, 220, 320, 420, 520) may use materials similar or commonly used as stent graft fabric material 16 including but not limited to DACRON, esPTFE with urethane, ePTFE, and others known in the art. The surface properties of the taper (if any) and the remaining portions of the dividing wall 20 (120, 220, 320, 420, 520) may be harder than that of the leading edge radius 22 (122, 222, 322, 422, 522). In addition, the remaining portions of dividing wall 20 (120, 220, 320, 420, 520) may be rougher than the leading edge radius 22 (122, 222, 322, 422, 522) for improved blood flow dynamics, possibly finding a biomimetic analogy in the roughness of a shark skin surface. Overall, the surface properties may be modified by coating procedures of additional materials such as nanomaterials and/or polymers. On the other hand, there may be no difference among the leading edge, wall and taper properties. The dividing wall 20 (120, 220, 320, 420, 520) may be attached on opposite sides to the stent graft body 11 using surgical sutures or any other strategy known in the art. The dividing wall 20 (120, 220, 320, 420, 520) in general, and the leading edge radius or rounded surface 22 (122, 222, 322, 422, 522) in particular could be strengthened with stent frame material (e.g., nitinol, CoCr, stainless steel, etc.) or maybe a stiffer PTFE skeleton arranged in such a way to allow crimping of the stent graft 10 within a delivery sheath in a conventional manner.

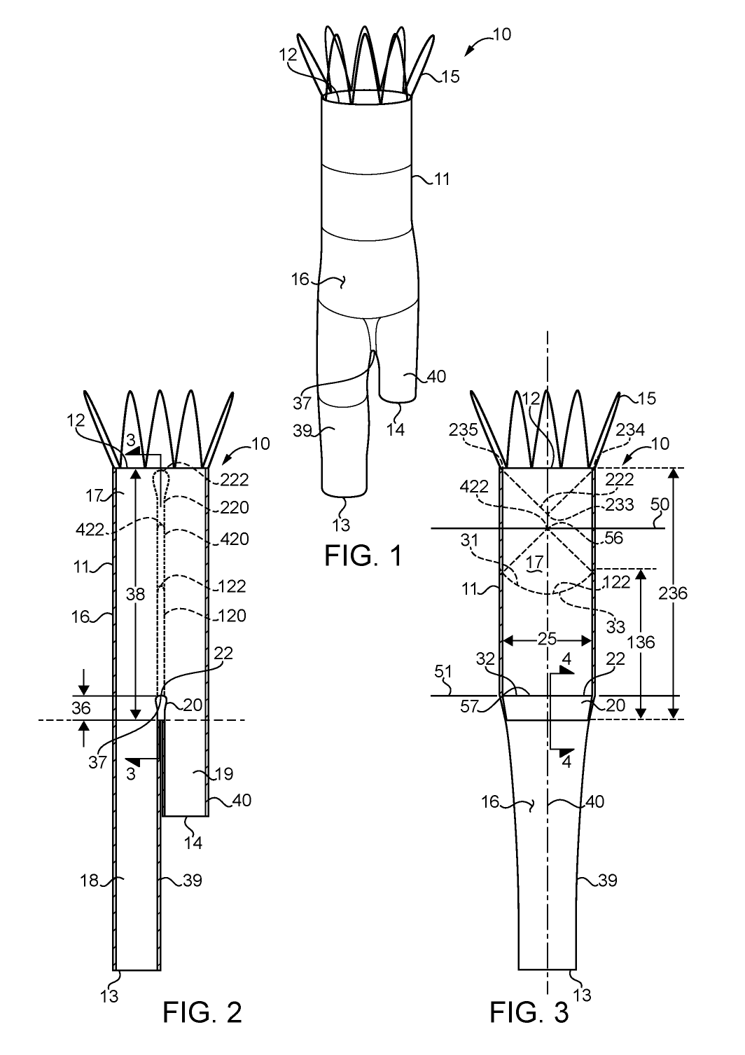

Referring now to FIGS. 9-15, a bifurcated stent graft 10 according to another aspect of the present disclosure is illustrated. This embodiment differs in that the dividing wall 520 terminates at a leading rounded surface 522 that is a portion of a bulb shape 48 that may improve blood flow characteristics analogous to maybe a bulbous bow that protrudes forward on some large ocean going ships. In the illustrated embodiment, as best shown in FIG. 14, bulb shape 48 may have a diameter 49 that is greater than a thickness 29 of the dividing wall 520 downstream from the bulb shape 48. As best shown in FIGS. 11 and 12, downstream from bulb shape 48, the dividing wall 520 may spread out in a swept fashion until reaching the side walls in contact with the stent graft fabric 16. Thus, this embodiment might be considered to have a convex leading edge radius. Unlike some of their previous embodiments, the leading rounded surface 522 of the bulb shape 48 extends less than a complete distance 45 between the graft fabric material on one side of the stent graft body 11 and the graft fabric material 16 on an opposite side of the stent graft body 11. This is to be contrasted, for instance with the embodiment of FIGS. 1-3 where the leading rounded surface 22 does extend the complete distance 45 between opposite sides of the stent graft body 11. In the embodiment of FIGS. 9-15, and as best shown in FIG. 14, a plane 50 perpendicular to a central axis of stent graft 10 is tangent to the leading rounded surface 522 at a point 56. This again is to be contrasted with the embodiment of FIGS. 1-3 in which the leading rounded surface 22 is tangent to a plane 51 perpendicular to the central axis at a line 57. In the case of the embodiment of FIGS. 9-15, the dividing wall 520 widens from the leading rounded surface 522 to the complete distance 45 over a transition segment 46 that extends a transition distance 47 along the central axis upstream from the bifurcation 37. Those skilled in the art will appreciate that the transition distance 47 determines how far the leading rounded surface of bulb shape 48 projects upstream from where the dividing wall 520 extends completely across the distance 45. In some instances it may be desirable to add one or more tethers 80 extending between the bulb shape 48 and the side wall of the stent graft 10. The stent graft 10 may include a plurality of tethers 80 that each have one end 81 attached to at least one of the stent 15 and/or graft fabric material 16, and an opposite end 82 attached to the dividing wall 522. The presence of tethers 80 may be desirable to both support the dividing wall and/or inhibit undesirable flow phenomenon such as flutter.

Referring again specifically to FIGS. 14 and 15, dividing wall 520 includes a cap 73 attached atop a partition 74. The cap 73, which includes the leading rounded surface 522, is of a material different from the partition 74. For instance, cap 73 may be formed of polyvinyl alcohol whereas the partition 74 may be constructed of PTFE. In particular, partition 74 may include an inner skeleton, maybe ePTFE for structural support. A leading edge core, which may define the shape of cap 73, and maybe even permeable esPTFE that permits some of the blood flow through dividing wall 520 to possibly account for pressure differential issues produced by anatomies and other factors in specific patients and applications. In one specific example, cap 73 may be formed of polyvinyl alcohol, which can be considered of biomimetic material, that mimics a hardness of a healthy aortic bifurcation. FIG. 15 is included to show the contrast between the thickness 529 of dividing wall 520 verses a wall thickness 43 of the stent graft material 16. The present disclosure contemplates dividing wall thicknesses 529 that is more than 1/20th of the wall thickness 43, however, the dividing wall thickness profile 529 may always be less than or equal to the wall thickness 43 of the stent graft material 16, especially when there is a desire to minimize the profile addition to when the stent graft is in a crimped state (not shown). These relative wall thickness teachings may also apply to all of the previous embodiments as well. The present disclosure prefers dividing walls that split the flow upstream from the bifurcation 37, but do not significantly add to the crimped state profile of the stent. FIG. 14 is also of interest for showing that the partition 74 may include an internal support skeleton 76 that is flanked by first and second wall layers 77, which may or may not exhibit some permeability to permit some blood flow through dividing wall 520 upstream from the stent graft bifurcation 37. The various components (cap 73, skeleton 76, wall layers 77) may be attached to each other in any suitable manner such as through electro spinning of nanobiocomposite blend fibers, ultrasonic welding of component layers, application of heat and/or compression, or any combination of these, or any other manner known in the art. The dividing wall 520 may be attached to the stent graft material 16 by sutures (e.g., prolene), or by any of the above described attachment methods. The dividing wall 520 may also or alternatively be attached to the stent struts directly without departing from the present disclosure.

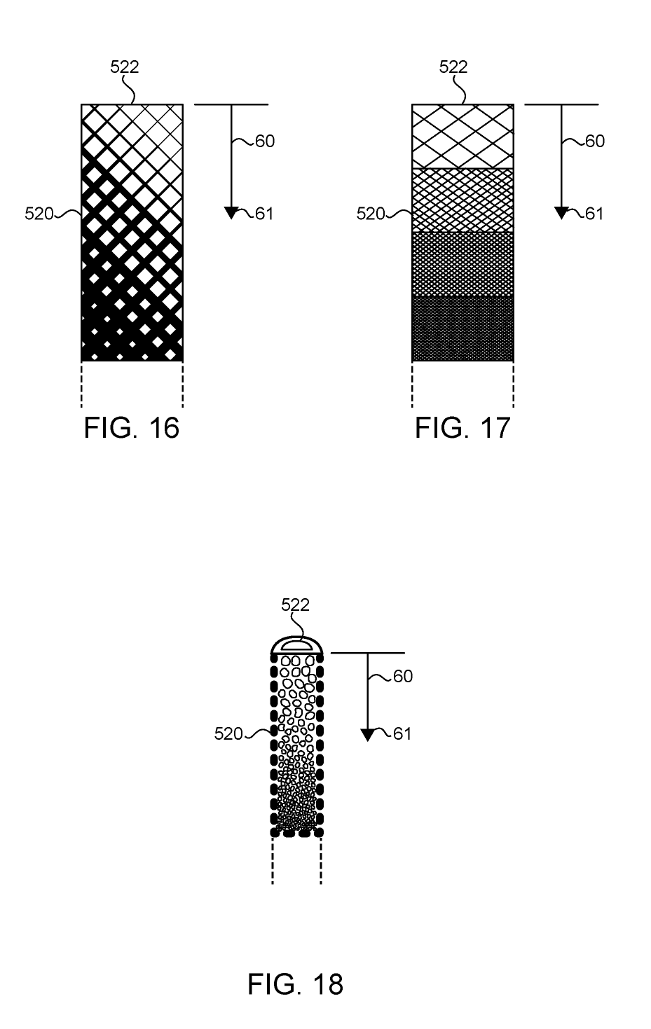

Referring now to FIGS. 16-18, several different strategies are illustrated for adding a permeability aspect to the dividing wall 520. Nevertheless, all of the previous embodiments could also include permeability in their respective dividing walls without departing from the present disclosure. Although a dividing wall with substantially uniform permeability in the direction toward the bifurcation 37 of the stent graft 10 is within the scope of this disclosure, FIG. 16-18 contemplate strategies in which the permeability of the dividing wall 520 changes from the leading rounded surface 522 toward the bifurcation 37 of the stent graft. In particular, the permeability of the dividing wall 520 may decrease with a distance 60 from the leading rounded surface 522 toward the bifurcation, which cannot be seen in the images of FIG. 16-18. This change in permeability may for instance be accomplished by a woven polyester material with a weave that tightens to decrease permeability in a direction 61 away from the leading rounded surface 522 toward the bifurcation 37 (not shown). FIG. 16 shows a continuum change in permeability, whereas FIG. 17 shows an example in which the change in permeability occurs step wise away from the leading rounded surface 522 so that different segments have uniform permeability but each segment presents a net decrease in permeability as one moves away from leading rounded surface 522. FIG. 18 is of interest for showing a sort of permeable pocket structure in which articles of different sizes of embedded material create a continuum of decreasing permeability in a direction 61 away from leading rounded surface 522. For instance, the embedded material could be a collagen based scaffold, with the pores in the collagen created by using directional freezing methods with consecutive freeze-drawing at varying cooling rates, as described in the literature. See for example Dieakar et al., and Anisotropic Freeze-Cast Collagen Scaffold For Tissue Regeneration: How Processing Conditions Affect Structure and Properties in the Dry and Fully Hydrated States, Journal of Mechanical Behavior of Biomedical Materials 90, 350-364, 219. Thus, in all cases of the stent graft according to the present disclosure having a permeable dividing wall, some blood can pass through the dividing wall 520 from one of the first and second flow paths 18 and 19 into the other one of the first and second flow paths. This strategy may be desirable, for instance to equalize pressures on both sides of the dividing wall.

### INDUSTRIAL APPLICABILITY

The present disclosure finds potential application in any bifurcated stent graft application. The present disclosure finds more particular applicability to bifurcated stent grafts for use in the blood circulatory system. Finally the present disclosure finds specific application in bifurcated stent grafts of the type used for abdominal aortic aneurism repair.

A bifurcated stent graft 10 according to the present disclosure may be delivered to a treatment site using known delivery devices and techniques. For instance, the bifurcated stent graft 10 might be compressed about a delivery catheter and covered by a retractable sheath, which is withdrawn at the delivery site to allow the stent graft 10 to self expand to the shape shown in FIG. 1. When the stent graft 10 expands at the delivery site, the previously compressed dividing wall 20 also expands into the configuration shown, for instance, in FIGS. 2 and 3. When this occurs, flow from the combined flow path 17 upstream from or at the main body opening 12 is divided into a first flow path 18 and a second flow path 19 at leading edge radius 22, which is spaced from an bifurcation 37 by dividing wall 20. The flow may transition from the leading edge radius 22 to a tapered segment 23 of the wall 20 at a tangent to the leading edge radius 22. Thereafter, the flow may transition from the tapered segment 23 to a uniform wall segment 24 prior to reaching one of the individual legs 39 and 40 of the bifurcated stent graft 10.

By appropriately choosing the thickness profile 21 of the dividing wall 20 and the radius r of the leading edge radius 22, less sheer stress may be encountered by blood components, potentially leading to less damage to blood cells and the like due to impact at the dividing point. Furthermore, by using an appropriate surface material and shapes, less or no damage may occur to blood components after the bifurcated stent graft 10 is in place. Furthermore, by making the leading edge radius 22 (122, 222, 322, 422, 522) soft like live tissue (i.e. biomimetic), less impact damage to blood cells could be expected. Permeability, if used, can help alleviate pressure differentials that may develop between the first and second flow paths 18 and 19 while the flow is being divided. The profile of the compressed stent graft 10 can be minimally increased by constructing the dividing wall to be relatively thin, compared to the thickness of the graft fabric material that makes up the bulk of the stent graft. The shape of the dividing wall, especially its leading rounded surface results in less or no damage to blood components, and all of the wetted stent graft surfaces are preferably chosen to reduce or eliminate damage to blood components.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A bifurcated stent graft comprising:
a stent graft body defining exactly one main body opening and two exit openings;
the stent graft body includes at least one stent attached to a graft fabric material, a bifurcation, and a dividing wall that divides a combined flow path into a first flow path and a second flow path that each terminate at one of the respective exit openings; and
the dividing wall includes a thickness profile that is equal to or thinner than a wall thickness of the graft fabric material, terminates at a leading rounded surface, and extends across a width of the combined flow path.

2. The bifurcated stent graft of claim 1 wherein the dividing wall is permeable to blood passing through the dividing wall from one of the first and second flow paths to an other one of the first and second flow paths.

3. The bifurcated stent graft of claim 2 wherein a permeability of the dividing wall to blood changes from the leading round surface toward the bifurcation.

4. The bifurcated stent graft of claim 3 wherein the permeability of the dividing wall decreases with a distance from the leading round surface toward the bifurcation.

5. The bifurcated stent graft of claim 4 wherein the dividing wall has a continuum of decreasing permeability in a direction from the leading rounded surface toward the bifurcation.

6. The bifurcated stent graft of claim 1 wherein the stent graft body includes a first leg and a second leg that each define one of the two exit openings; and
a stretchable membrane attached to, and extending between, the first leg and the second leg.

7. The bifurcated stent graft of claim 6 wherein the stretchable membrane has an aortic bifurcation contact surface that is a separation distance from the bifurcation of the stent graft body.

8. The bifurcated stent graft of claim 6 wherein the stretchable membrane is oriented perpendicular to the dividing wall.

9. The bifurcated stent graft of claim 1 wherein the stent graft body defines a central axis;
the leading rounded surface of the dividing wall extends less than a complete distance between the graft fabric material on one side of the stent graft body and the graft fabric material on an opposite side of the stent graft body.

10. The bifurcated stent graft of claim 9 wherein the dividing wall widens from the leading rounded surface to the complete distance over a transition segment that extends a transition distance along the central axis upstream from the bifurcation.

11. The bifurcated stent graft of claim 9 wherein a plane perpendicular to the central axis is tangent to the leading rounded surface at a point.

12. The bifurcated stent graft of claim 9 wherein a plane perpendicular to the central axis is tangent to the leading rounded surface at a line.

13. The bifurcated stent graft of claim 9 wherein the leading rounded surface is a portion of a bulb shape with a diameter that is greater than a thickness of the dividing wall at a location downstream from the bulb shape.

14. The bifurcated stent graft of claim 1 wherein dividing wall includes a cap attached atop a partition; and
the cap, which includes the leading rounded surface, is of a material different from the partition.

15. The bifurcated stent graft of claim 14 wherein the cap is formed of a biomimetic material that mimics a hardness of an aortic bifurcation.

16. The bifurcated stent graft of claim 1 wherein the thickness profile of the dividing wall is more than 1/20 the wall thickness of the graft fabric material.

17. The bifurcated stent graft of claim 1 wherein the dividing wall includes an internal support skeleton that is flanked by first and second wall layers.

18. The bifurcated stent graft of claim 1 including a plurality of tethers that each have one end attached to the dividing wall and an opposite end attached to at least one of the stent and the graft fabric material.

19. A bifurcated stent graft comprising:
a stent graft body defining exactly one main body opening and two exit openings;
the stent graft body includes at least one stent attached to a graft fabric material, a bifurcation, and a dividing wall that divides a combined flow path into a first flow path and a second flow path that each terminate at one of the respective exit openings;
the dividing wall includes a cap attached atop a partition; and
the cap is of a material different from the graft fabric material, extends across a width of the combined flow path, and is formed of a biomimetic material that mimics a hardness.
